(19) United States
(12) Patent Application Publication
(10) Pub. No.: US 2026/0348869 A1
(43) Pub. Date: Nov. 5, 2026

(54) MEASUREMENT SYSTEM FOR AND MEASUREMENT METHOD OF MOVING WEB OF TISSUE PAPER

(71) Applicant: VALMET AUTOMATION OY, Espoo (FI)

(72) Inventors: Janne PAASO, Espoo (FI); Pekka SUOPAJÄRVI, Espoo (FI); Markku MÄNTYLÄ, Espoo (FI)

(73) Assignee: VALMET AUTOMATION OY, Espoo (FI)

(21) Appl. No.: 19/667,255

(22) Filed: May 4, 2026

(30) Foreign Application Priority Data
May 5, 2025 (FI) 20253175

(51) Int. Cl. G01N 21/89; G01N 21/3559

(52) U.S. Cl. CPC G01N 21/8914; G01N 21/3559; G01N 21/8903; G01N 2021/8917

(57) ABSTRACT

A measurement system for measuring a moving web of tissue paper comprises an optical sensor performing optical transmission measurements of absorption of said moving web within at least two of the following ranges: shorter than 1.4 μm, 1.6 μm to 1.9 μm, longer than 2.15 μm, which include an absorption characteristic of a desired material component of the web and two references for the absorption characteristic. The optical sensor enables detection of at least thirteen spectral elements distributed over the optical ranges in a manner independent of both the at least one absorption characteristic and the at least two references. The optical sensor distinguishes spectral elements associated with the absorption characteristic and the reference from each other. The optical sensor traverses across the moving web. A data processing unit processes data on at least three of at the at least thirteen spectral elements, and forms information on at least one of the following based on the data on the spectral elements: a basis weight of the web and a weight per unit area of at least one of the at least one desired material component of the web.

### FIELD

The invention relates to a measurement system for and a measurement method of a moving web of tissue paper.

### BACKGROUND

Moisture and cellulose content of a web of paper or board may be measured by directing infrared light from an optical source to the web. Interaction between the web and the infrared light that travels through the web attenuates the power of the infrared light. Water and cellulose absorb strongly at known, narrow optical peaks in infrared regions. Strengths of the wavelength dependent absorptions are related to the amount of water and cellulose in the web. When the absorption of water or cellulose is measured, it is possible to determine moisture content or cellulose content of the web, respectively. They together can be used to define basis weight of the web.

The optical measurement of the basis weight depends on a particle size distribution of the web which causes variations in the results when webs of the same basis weight, but different particle size distributions are measured. Additional variation may be caused by other properties of webs, which may come from chemistry, for example. Hence, there is a need for improvement in the measurements.

### BRIEF DESCRIPTION

The present invention seeks to provide an improvement in the measurements.

The invention is defined by the independent claims. Embodiments are defined in the dependent claims.

If one or more of the embodiments is considered not to fall under the scope of the independent claims, such an embodiment is or such embodiments are still useful for understanding features of the invention.

### LIST OF DRAWINGS

Example embodiments of the present invention are described below, by way of example only, with reference to the accompanying drawings, in which

FIG. 1 illustrates an example of an optical sensor;

FIG. 2A illustrates an example of a plurality of optical sources that output spectral elements to a plurality of detectors of the optical sensor;

FIG. 2B illustrates an example of spreading different spectral elements to different detectors of the optical sensor;

FIG. 2C illustrates another example of spreading optical radiation through filters to different detectors of the optical sensor;

FIG. 2D illustrates an example of non-mechanical sweep of spectral elements;

FIG. 3 illustrates an example of attenuation of optical radiation in interaction with a web of paper or board;

FIG. 4 illustrates an example of a paper machine;

FIG. 5 illustrates an example of traversing measurement device;

FIG. 6 illustrates an example of a data processing unit;

FIG. 7 illustrates an example of variation of spectrum with different material components and example wavelengths for optical measurements; and

FIG. 8 illustrates of an example of a flow chart of a measuring and control method.

### DESCRIPTION OF EMBODIMENTS

The following embodiments are only examples. Although the specification may refer to “an” embodiment in several locations, this does not necessarily mean that each such reference is to the same embodiment(s), or that the feature only applies to a single embodiment.

Single features of different embodiments may also be combined to provide other embodiments. Furthermore, words “comprising” and “including” should be understood as not limiting the described embodiments to consist of only those features that have been mentioned and such embodiments may also contain features/structures that have not been specifically mentioned. All combinations of the embodiments are considered possible if their combination does not lead to structural or logical contradiction.

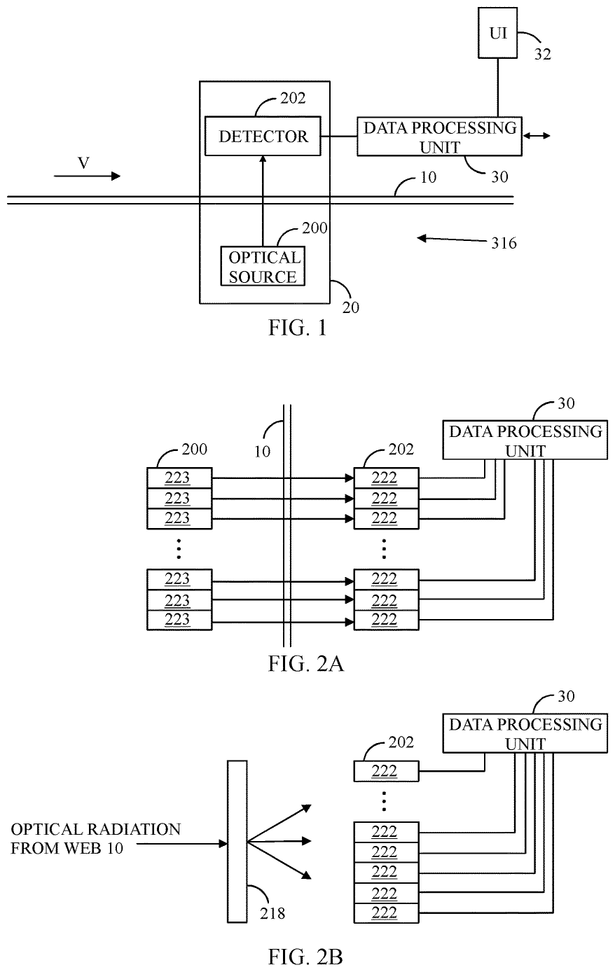

FIG. 1 illustrates a measurement system for measuring a moving web 10 of tissue paper. The movement of the web 10 is illustrated by the letter V and an arrow. The measurement system comprises at least one optical sensor 20 that performs optically transmission measurements of absorption of the web 10. The optical sensor 20 comprises at least one optical source 200 and a detector arrangement 202. The optical source 200 may include one or more units and the detector arrangement 202 may include one or more detectors 222. The measurement system may utilize an optical range about 1 μm to about 2.5 μm, for example. The lower limit may be shorter than 1 μm and/or the upper limit may also be a longer wavelength than 2.5 μm. The optical range covers at least one absorption characteristic of at least one desired material component of the web 10 and at least two references for the at least one absorption characteristic.

The material components that are wished to be measured may be called desired material components. That means, all or only a part of the material components of the web 10 may be measured.

Each absorption characteristic is a value derived from a relative intensity, which indicates the percentage of an initial intensity that passes through the web 10. This way, the relative intensity represents a comparison between the initial and transmitted intensity. Each absorption characteristic refers to an absorption value that is clearly different from an average value of the optical range. An absorption characteristic refers to a local absorption maximum i.e. a peak within the optical range, and its location within the optical range is at least almost unique to a desired material component of the web 10. Usually, a certain material component causes one or more local maximums which can be reliably used to characterize a percentage of the material component within the web 10. That is, the one or more absorption characteristics are distinctive attributes of the desired material components of the web 10. A person skilled in the art is familiar with absorption and absorption peaks, per se.

The optical detector arrangement 202 performs, in an optical range, optical transmission measurements of absorption of the web 10 including at least two of the following three ranges: the wavelengths are equal to or shorter than about 1.4 μm, an optical range about 1.6 μm to about 1.9 μm and an optical range where the wavelengths are equal to or longer than about 2.15 μm. That is, the overall measurement range is defined by at least two of the three ranges. Thus, the measurements may be performed in the range shorter than about 1.4 μm and the range about 1.6 μm to about 1.9 μm. Alternatively, the measurement may be performed in the range shorter than about 1.4 μm and the range where the wavelengths are equal to or longer than about 2.15 μm. Still alternatively, the measurement may be performed in the range about 1.6 μm to about 1.9 μm and the range where the wavelengths are equal to or longer than about 2.15 μm.

These three ranges cover at least one absorption characteristic of at least one desired material component of the web 10 and at least two references for the at least one absorption characteristic. The at least two references may be used to define a baseline which may be illustrated as a straight line or a curve with or without tilt. With more than two references, the baseline that is a curve instead of a straight line may be determined.

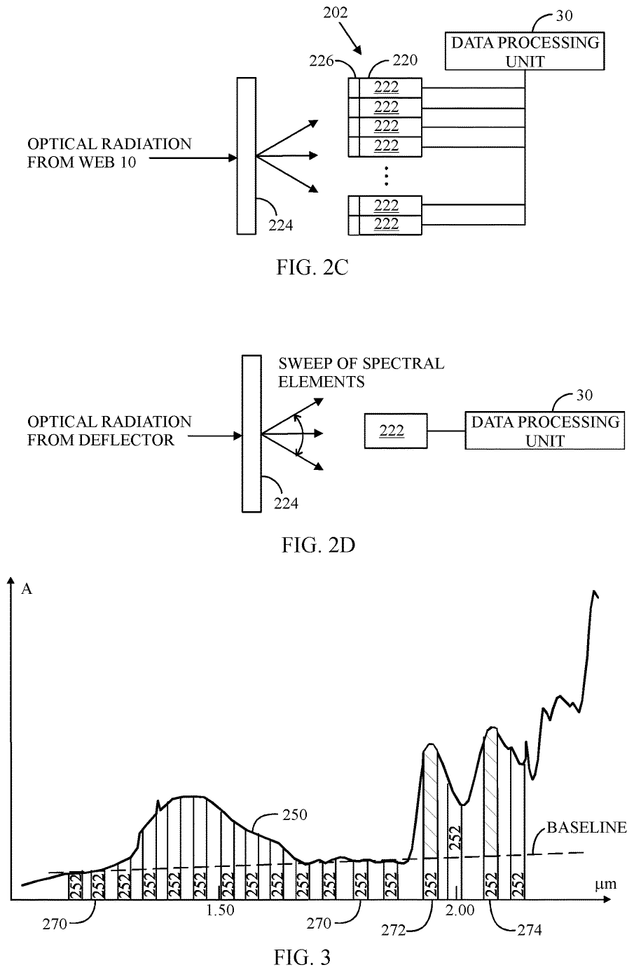

The optical detector arrangement 202 can perform detections of at least thirteen mutually independent spectral elements 252 that are distributed over the optical ranges of the measurement system regularly or irregularly (FIG. 3 illustrates the spectral elements 252). The optical detector arrangement 202 may have detectors 222 for each measurement of the spectral elements 252 (FIGS. 2A to 2C illustrate the detectors). The spectral elements 252 can be considered sections or segments of the optical range. The spectral elements 252 may be relatively narrow and they may correspond one-to-one with detectors 222 in FIG. 2A. In that manner, the number of the detectors 222 may define the number of the spectral elements 252. The spectral elements 252 may be non-overlapping, and at least only partially overlapping if they are overlapping at all. The spectral elements 252 are distributed over the optical range in a manner independent of both the at least one absorption characteristic and the at least two references of said at least one desired material component.

The number of the spectral elements 252 of the optical range is equal to or larger than a combined number of the at least one absorption characteristic of the at least one desired material component and the at least one reference. In this manner, the optical detector arrangement 202 can distinguish the spectral elements 252 associated with the at least one absorption characteristic of the at least one desired material component and the at least two references from each other.

A data processing unit 30 receives and processes data on the repeated measurements each performed at the at least thirteen independent spectral elements 252. The data processing unit 30 forms information on at least one of the following based on the data on at least three of the at least thirteen independent spectral elements 252: a basis weight of the web 10 and a weight per unit area of at least one of the at least one desired material component of the web 10. That means, the data processing unit 30 does not need to utilize all the potential information from the at least thirteen spectral elements 252. Only three spectral elements 252 may be utilized at minimum. In such a case, the first and second spectral elements 252 may be used for determining the baseline and the third spectral element 252 may be used for determining an absorption characteristic of the web 10.

Because the number of the spectral elements 252 of the optical range is equal to or larger than a combined number of the at least one absorption characteristic of the at least one desired material component and the at least two references, the data processing unit 30 can distinguish the spectral elements 252 associated with the at least one absorption characteristic of the at least one desired material component and the at least two references from each other. Because the detections are performed at a large number of spectral elements 252, the spectral elements 252 do not need to be directly linked to any specific absorption characteristic or wavelength that may be desired to be measured from the web 10. The number of the spectral elements 252 and their density enables successful measurements of any desired material component that has technically useful response within the optical range. Still, the number of the spectral elements 252 can be low enough for a simple measurement.

In an embodiment examples of which are illustrated in FIGS. 2A to 2B, the optical detector arrangement 202 detects simultaneously the at least thirteen independent spectral elements 252 that are non-overlapping. The term non-overlapping means that the spectral elements 252 do not over-lap at all or they are only partially over-lapping. Namely, the partial over-lapping that causes a resolvable spectral difference between the detectors 222 or detections may be allowed. A person skilled in the art is familiar with that kind of feature, per se, the feature relating to tolerance and/or resolution of the detectors 222.

In an embodiment, an example of which is illustrated in FIG. 2A, the optical source 200 of the optical detector arrangement 202 comprises a plurality of optical source units 223, each of which outputs a single spectral element 252 as a beam of light. The beams of light of the spectral elements 252 travel through of the web 10, and different detectors 222 of the detector arrangement 202 receive the different spectral elements 252 such that each of optical detectors 222 receives only one spectral element 252 while all optical detectors 222 receive spectral elements 252. The number of the optical detectors 222 is equal to or larger than the number of the spectral elements 252. The number of the optical source units 223 is equal to or larger than the number of the spectral elements 252 in this example. In that manner, the optical source units 223 and the detectors 222 can be paired. Note that the optical detectors 222 may receive also other beams if they are output by the optical source 200. In an embodiment an example of which is illustrated in FIG. 2B, the optical detector arrangement 202 comprises an array 220 of detectors 222. The number of the optical detectors 222 is equal to or larger than the number of the spectral elements 252. In this example, the optical source 200 may be a wide band source that outputs a plurality of spectral elements 252. The optical detector arrangement 202 may direct different spectral elements 252 of the optical radiation to different detectors. A dispersive component 218 may direct beams of light of different spectral elements 252 at different angles based on their wavelengths. The dispersive component 218 may comprise one or more prisms and/or one or more gratings, for example. Each spectral element 252 may then be detected by one detector 222. Different detectors 222 then receive the beams of light of different wavelengths including the spectral elements 252.

In an embodiment, an example of which is illustrated in FIG. 2C, the optical detector arrangement 202 may comprise an electrically controllable optical beam deflector 224. The electrically controllable optical beam deflector may comprise an acousto-optic beam deflector and/or a magneto-optic beam deflector. For each spectral element 252, there may be optical filters 226 between the electrically controllable optical beam deflector 224 and the detectors 222. In that manner, a single filter of the optical filters 226 passes one of the spectral elements 252 to a unique detector 222, and that leads to an operational structure where each of the spectral elements 252 is detected by one of the detectors 222 based on the filters and the wavelengths they allow to travel through. The filters 226 may be notch filters, for example. These optical deflectors are so fast that the detections and the required data processing for the measurement can be performed in real time and the detections can be considered simultaneous from the point of process measurements and control. That kind of simultaneity or simultaneousness, per se, is known both in control systems and by a person skilled in the art. Note also that the at least two measurements may also be averaged, and it is still enough for a real time measurement. The web 10, although moving, for example, 27 m/s or less, can move less than about a millimeter in the period within which all at least thirteen detections of the spectral elements have been made. That is, the optical deflector's operational frequency can be 500 kHz or less, for example.

FIG. 2D illustrates an example of non-mechanical sweep of spectral elements. In this kind of embodiment, the number of detectors 222 may be smaller than the number of the spectral elements 252 (FIG. 2D shows only one detector 222). All the spectral elements 252 may be detected such that different spectral elements 252 are swept non-mechanically over the detector(s) 222. In an embodiment, spectral elements 252 may be swept over only one detector 222. The non-mechanical sweeping may be performed using electro-optical and/or magneto-optical deflectors, for example (see FIG. 2C, that shows some examples of such devices). A person skilled in the art is familiar with various sweeping possibilities and non-mechanical deflectors.

In embodiments examples of which are illustrated in FIGS. 2A to 2D, the optical detector arrangement 202 may enable the detections of the at least thirteen independent spectral elements 252 that are non-overlapping at least almost simultaneously or in real time.

In an embodiment, the optical detector arrangement 202 may have detectors 222 of the array 220 selected in a non-mechanical manner for the detections of the desired material components. Additionally or alternatively, the data processing unit 30 may activate the detectors 222 for detecting only the spectral elements 252 associated with at least one absorption characteristic of cellulose and at least one absorption characteristic of water, and the at least one reference of cellulose weight and water weight. The independent spectral elements 252 may be selected as a function of the wavelengths of said optical range based on the desired material components to be measured.

In an embodiment, the data processing unit 30 may activate detectors 222 of the array 220 electrically for detection and data processing. In that manner, the detectors 222 that are needed to detect the absorption characteristics of any material component of the web 10 and their references can be selected and/or activated and those that are not necessarily needed for detections of absorption characteristics and/or reference may be deactivated. When composition of the web 10 changes, it is possible to activate different detectors 222 for detecting different spectral elements 252. In that manner, the detector arrangement 202 does not need to be changed but a reprogramming is enough for a different or varying web 10. In general, at least two measurements may be averaged for limiting variation of the values of the measurements.

FIG. 3 illustrates an example of absorption curve 250 of the web 10 and the spectral elements 252 that are distributed over the overall optical range of the measurement, which in this example is about 1.2 μm to about 2.2 μm. The y-axis is attenuation or absorbance in an arbitrary scale. FIG. 3 relates to the web 10 of a paper and board industry but it is easy to generalize to any web 10 such as that of a nonwoven fabric. The spectral elements 252 may be distributed regularly or irregularly. In FIG. 3, which is a mere example, the spectral elements 252 are distributed almost regularly but as can be seen there are also slight irregularities for illustrating also them. That is, the distance between consecutive spectral elements 252 can be constant if the distribution is regular. If the distribution is irregular, the distance between consecutive spectral elements 252 is not constant. The distance can be defined to be measured between the middle points of each spectral element 252, for example. Additionally, different spectral elements 252 may have varying widths instead of a constant width in an embodiment. The spectral elements 252 are distributed in an independent manner of both the absorption characteristics 272, 274 and the at least one reference 270 of the material components. In that manner, the same measurement system can be used for various webs 10 that may have one or more different compositions of material components. A number of spectral elements 252 of the optical range of the measurement is equal to or larger than a combined number of the absorption characteristics of the desired material components and the references.

Note that the spectral elements 252 are not designed to detect any particular absorption characteristic. The distribution and the number of the spectral elements 252 in the distribution enables the measurement of the absorption characteristics of interest because at least one of the spectral elements 252 is at or measurement technically close enough to any absorption characteristics. That means, although a spectral element 252 and an absorption characteristic do not necessarily overlap for an actual measurement of the absorption characteristic as taught in handbooks and/or known by a person skilled in the art, they are close enough to give a meaningful result for the information on the web 10 and its materials.

In paper processing, spectral elements 270, 272, 274 among all the spectral elements 252 may be utilized. The x-axis is wavelength in micrometers. Water has an absorption characteristic in the spectral element 272 that is at about 1.95 μm (wavenumber about 5130 cm−¹), for example, and thus moisture percentage or water mass per unit area of the web 10 may be measured at the spectral element 272. Cellulose, which is a material component of the web 10, has an absorption characteristic in the spectral element 274 that is at about 2.11 μm (wavenumber about 4740 cm−¹), for example, and thus cellulose mass per unit area of the web 10 may be at the spectral element 274. Absorption of the optical radiation that is considered to be similar to that of the absorption characteristics takes place at and/or at about a wavelength where a local maximum of absorption is. The peaks of the spectral elements 272, 274 of absorption characteristics are local maximums soaring above their environment that is detected.

Reference measurements to determine the relative water absorption and the relative cellulose absorption should be measured at spectral elements where no desired material components have characteristic absorption. Such non-characteristic absorption spectral elements 270 i.e. references may be at about 1.8 μm (wavenumber about 5555 cm⁻¹) and about 1.3 μm (wavenumber about 7692 cm⁻¹), for example, without limiting to these. A baseline, which the person skilled in the art is, per se, familiar with, may be determined with the reference measurements. The baseline defines a general tilt of slope of the attenuation as a function of the wavelength. The hatched area above the baseline at the dominant wavelengths represent strength of attenuation or absorption. Note the baseline may but does not need to be a straight line.

The absorption characteristic of talcum may be measured at about 1390 nm. The absorption characteristic of clay may be measured at about 2208 nm. The absorption characteristic of calcium carbonate may be measured at about 3980 nm, for example.

In an embodiment, a number of the spectral elements 252 of the optical range is equal to or larger than a combined number of the at least one absorption characteristic of the at least one desired material component and the at least two references. The number of the spectral elements 252 may be the same as the number of the detectors 222 that data processing unit 30 may actively utilize. The data processing unit 30 may activate and deactivate the detectors 222 for performing detections associate with the at least one absorption characteristic of the desired material components.

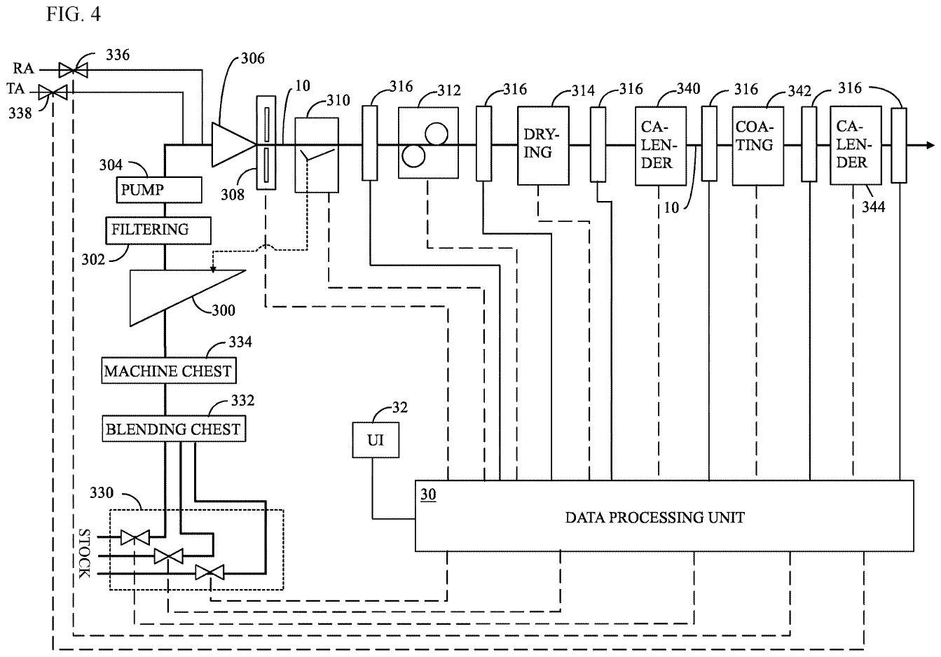

Examine an example of a paper machine illustrated in FIG. 4. One or more stocks, which may include ash, cellulose and/or moisture as material components of the web 10, are fed onto a paper machine through a wire pit silo 300, which is usually preceded by a blending chest 332 for partial stocks and a machine chest 334. The machine stock is dispensed for a short circulation, for instance, controlled by a basis weight control or a grade change program. The blending chest 332 and the machine chest 334 may also be replaced by a separate mixing reactor (not shown in FIG. 4), and the dispensing of the machine stock is controlled by feeding each partial stock separately by means of valves or another flow control means 330. In the wire pit silo 300, water is mixed into the machine stock to obtain a desired consistency for the short circulation (dashed line from a former 310 to the wire pit silo 300).

From the obtained stock it is possible to remove sand or the like (centrifugal cleaners), air (deculator) and other coarse material (pressure filter) using cleaning devices 302, and the stock is pumped with a pump 304 to a headbox 306. The sand or the like that avoids removal may form a part of ash in paper or board. Before the headbox 306, it is possible to add to the stock, in a desired manner, a filler TA, which includes one or more material components of the web 10, including e.g. gypsum, kaolin, calcium carbonate, talcum, chalk, titanium dioxide and diatomite etc. and/or a retention agent RA, which also includes one or more material components of the web 10, such as inorganic, inartificial organic or synthetic water-soluble organic polymers. The filler TA and/or the retention agent RA may include ash as a material component of the web 10. A person skilled in the art is familiar with fillers and retention agents, per se.

Ash is what remains of paper or board after combustion at 900 °C according to TAPPI T 413 “Ash in Wood, Pulp, Paper and Paperboard: Combustion at 900 °C”. According to TAPPI T 211 om-02 , igniting papers or pulp containing no added fillers or coatings at either 525 °C or 900 °C will yield essentially identical results of a few tenths percent ash or less.

According to the standard (TAPPI T 211 om-02 ), ash may include various residues from chemicals used in paper or board manufacture, metallic matter from piping and machinery, mineral matter in the pulp from which the paper was made, and/or filling, coating, pigmenting and/or other added materials. In general, ash is material of paper or board that does not combust. All these substances can be considered material components of the web 10.

From the headbox 306 the stock is fed through a slice opening 308 of the headbox to a former 310, which may be a fourdrinier wire or a gap former. In the former 310, water drains out of the web 10 and additionally ash, fines and fibres are led to the short circulation. In the former 310, the stock is fed as a web 10 onto a wire, and the web 10 is preliminary dried and pressed in a press 312. The web 10 is actually dried in driers 314. In general, there is at least one pair of sensors 316 that performs the optical measurement and potentially an X-ray measurement for determining basis weight and/or dry stuff content of the web 10. The at least one pair of sensors 316 feeds the measured data to a data processing unit 328 (see continuous lines therebetween). The basis weight may be considered the same as the dry stuff content if water weight is not taken into account. Additionally, the basis weight may be called grammage.

The paper or board machine may also include a pre-calender 340, a coating section 342 and/or a finishing calender 344. It is not necessary to have the coating section 342, however, and therefore it is not necessary to have more calenders 340, 344 than one. In the coating section 342, coating paste, which includes one or more material components may include ash, may be spread onto paper. The coating paste may include gypsum, kaolin, talcum or carbonate, starch, latex or the like as material components of the web 10, for example. A person skilled in the art is familiar with the coating paste and its substances, per se.

In calenders 340, 344, where the uncoated or coated paper or board web runs between the rolls pressing with desired force, it is possible to change the surface properties of the paper, such as smoothness, roughness, topography, gloss and the like. The calender 340, 344 may also affect the paper thickness and/or the basis weight or other mass per unit area of the paper or board, for example. In the calender 340, 344, the properties of the paper web may be changed by means of web moistening, temperature and nip pressure between the rolls. In addition to this, it is clear that the operation of a paper machine is known, per se, to a person skilled in the art, and therefore, it need not be presented in greater detail in this context.

FIG. 4 also shows a control arrangement of the paper machine. Then the data processing unit 30 may also perform the data processing for the control of the paper or board machine based on the measurements of the web 10. Factors affecting the quality and grade change include, inter alia, the number and mutual proportion of partial stocks, the amount of filler, the amount of retention agent, machine speed, the amount of white water and drying capacity. These may include the desired materials of the web 10. The data processing unit 30 may control the dispensing of partial stocks by means of valves 330, the dispensing of each filler TA by means of the valve 338, the dispensing of the retention agent RA by means of the valve 336, adjust the size of the slice opening 308, control the machine speed, control the amount of white water and the drying process in block 314 (see dashed lines). The data processing unit 30 utilizes the sensors 316 for measuring the web 10. The data processing unit 30 may also measure the properties of the web 10 elsewhere (e.g. at the same locations where controls are performed). The sensor(s) 316 of FIG. 4 can be considered to include the sensor 20 of FIG. 1.

The data processing unit 30 may be conceived as a paper machine's control arrangement, or part thereof, based on automatic data processing. The data processing unit 30 may receive digital signals or convert the received analog signals to digital ones. The data processing unit 30 may comprise at least one processor and at least one memory and execute the signal processing in accordance with an appropriate computer program. The operating principle of the data processing unit 30 may be, for instance, PID (Proportional-Integral-Derivative), MPC (Model Predictive Control) or GPC (General Predictive Control) control.

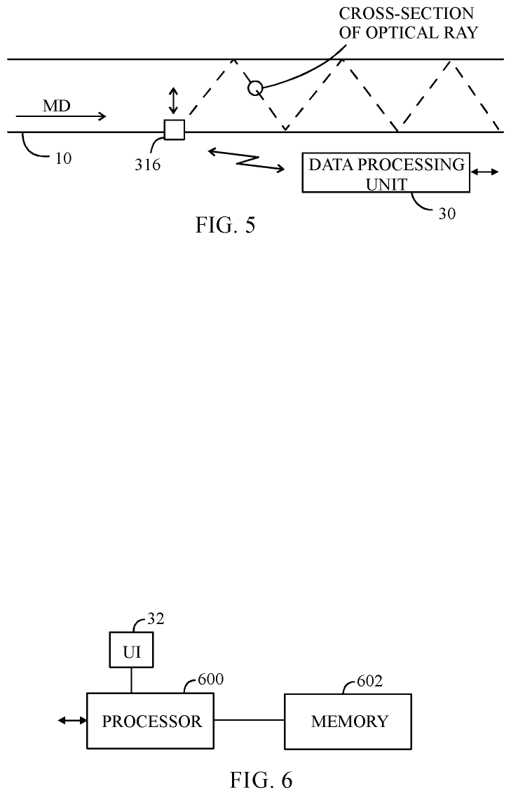

In an embodiment illustrated in FIG. 5, the measurement apparatus comprises the sensor 316 that traverses over the moving web 10 in a cross direction that is perpendicular to the machine direction. The sensor 316 may comprise at least one of the optical sensors 20. FIG. 5 illustrates the traverse with a zig-zag dashed line. When the web 10 moves in the machine direction and the sensor 316 travels in a perpendicular direction that makes the line of measurement to become zig-zag over the web 10. The communication between the traversing sensor 316 and the data processing unit 30 may be wired or wireless which is also shown in FIG. 5.

In an embodiment, the optical sensor 20 may measure the spectral elements 252 associated with the at least one absorption characteristic of the at least one desired material component of the web 10 that comprises one or more solid material components in a fiber form. The optical range then includes one or more absorption characteristics of the at least one solid material component of the web 10 and at least one reference for the optical absorption characteristics. The data processing unit 30 may consequently form the basis weight, and the weight per unit area of the at least one solid material component of the web 10.

In an embodiment, the system may measure the at least one absorption characteristic of the web 10 that comprises a nonwoven fabric.

In an embodiment, the optical sensor 20 may measure the spectral elements 252 associated with the at least one absorption characteristic of the desired material components of the web 10 that comprise water, cellulose and one or more ash components. The web 10 can then be considered a paper or board web. The optical range then includes the at least one absorption characteristics of the following: water, cellulose and at least one or more ash components of the web 10. Based on these, the data processing unit 30 forms the basis weight, the water weight per unit area, the cellulose weight per unit area and at least one weight per unit area of the one or more ash components of the web 10.

In an embodiment, the optical sensor 20 may measure the spectral elements 252 associated with the at least one absorption characteristic of the at least one material component that is solid and includes at least one of the following: retention agent material, polymer material, monomer material, filling material, coating material and pigmenting material.

In an embodiment, the optical sensor 20 may measure the spectral elements 252 associated with the at least one absorption characteristic of the at least one ash component that includes at least one of the following: talcum, kaolin, gypsum, starch, latex, calcium carbonate, titanium oxide.

In an embodiment, the optical sensor 20 may measure the spectral elements 252 associated with the at least one absorption characteristic of fossil-based polymer fibers, bio-based polymer fibers and/or modified polymer fibers of the web 10.

FIG. 6, illustrates an example of the data processing unit 30. The data processing unit 30 may comprise one or more processors 600, and one or more memories 602 including computer program code. The one or more memories 602 and the computer program code may, with the one or more processors 600, cause the data processing unit 30 at least to determine basis weight of the paper or board web 10 based on the data on at least three spectral elements of the at least thirteen spectral elements 252 associated with absorption characteristics of water, cellulose, at least one material component of the web 10 and at least one reference, the absorption characteristics being based on the detections distributed over the optical range of the infrared light.

A user interface 32 means an input/output device and/or unit. Non-limiting examples of a user interface include a touch screen, other electronic display screen, keyboard, mouse, microphone, and handheld electronic controller.

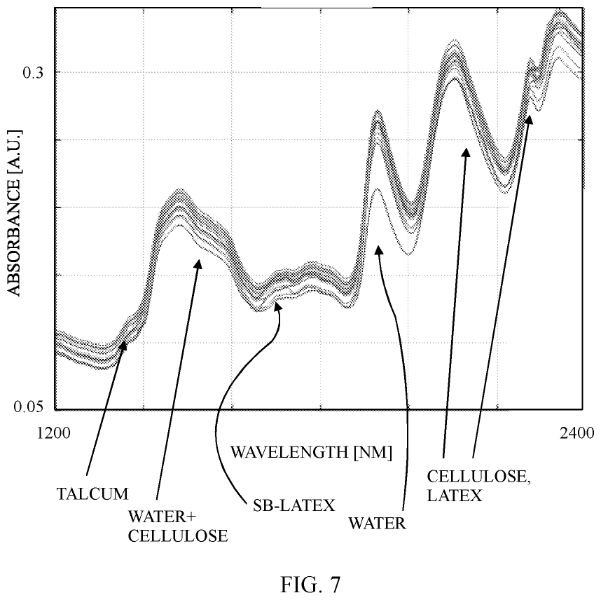

FIG. 7 illustrates an example of variation of absorbance with different material components of the web 10 and example wavelengths for optical measurement. Examples of dominant absorption peaks relate to talcum, water, cellulose and SB-latex (Styrene-Butadiene latex). A person skilled in the art is familiar with dominant absorption peaks of material components that are desired to be measured. A person skilled in the art is also familiar with material components that are used in various webs 10 and their optical features.

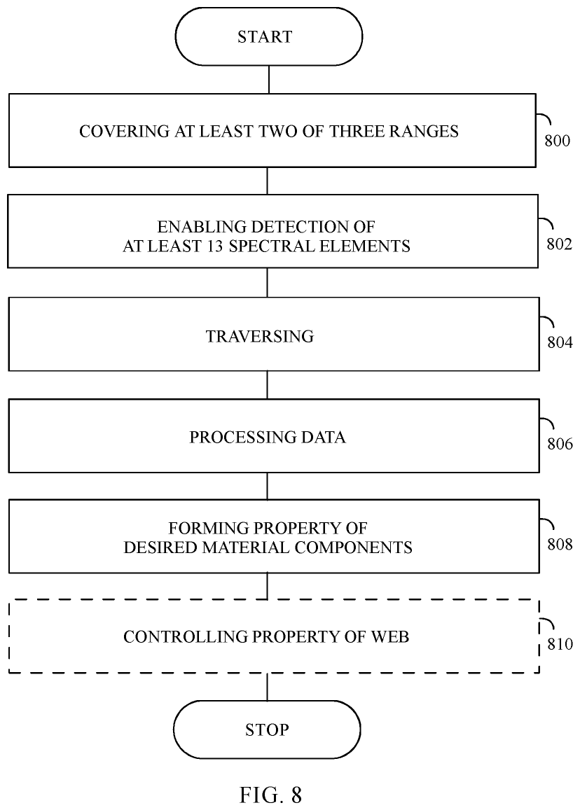

FIG. 8 is a flow chart of the measurement method of a moving web 10. In step 800, an optical range of at least two of the following ranges of the method is covered by an optical sensor 20: equal to or shorter than about 1.4 μm, about 1.6 μm to about 1.9 μm, and equal to or longer than about 2.15 μm, which include at least one absorption characteristic of at least one desired material component of the web 10 and at least two references for the at least one optical absorption characteristic.

In step 802, detection 802 is enabled by the optical sensor 20 that performs transmission measurements of at least thirteen independent spectral elements 252 that are distributed over the optical ranges of the method regularly or irregularly in a manner independent of both the at least one absorption characteristic and the at least one reference of said desired material components for enabling the optical sensor 20 to distinguish spectral elements 252 associated with the at least one optical absorption characteristic and the at least two references of the at least one desired material component from each other.

In step 804, the optical sensor 20 transverses across the moving web 10 in a transverse direction of the web 10.

In step 806, data on at least three of the thirteen independent spectral elements 252 is processed 806 by a data processing unit 30.

In step 808, information on at least one of the following based on the data on the at least three of the at least thirteen independent spectral elements 252 associated with absorption is formed by the data processing unit 30: a basis weight of the web 10 and a weight per unit area of at least one of the desired material components of the web 10.

In an embodiment an example of which is illustrated in FIG. 4, the basis weight and the weight per unit area of the at least one material component of the web 10 is controlled.

In an embodiment, a measurement of a multilayer web 10 is performed. In an embodiment, a baseline curvature of the optical range is formed, and the basis weight based on absorptions of the infrared light of the detected elements relating to cellulose and water and the baseline curvature is determined. The curvature and/or tilt has an effect on the absorption values because the peak of the absorption may be too high without taking into account the baseline curvature. That can be seen in FIG. 3. Namely, if the baseline were a horizontal line, i.e. the slope were 1, the peaks of dominant absorption close to 2 μm would appear higher than with the correction of the baseline with a positive slope as a function of the wavelength that is shown with a dashed line in FIG. 3. Note also that with different reference measurements, the baseline may also have a negative slope. Then some peaks may appear lower than they actually are. That is why the peaks should be measured with respect to the estimated baseline at that wavelength.

It will be obvious to a person skilled in the art that, as technology advances, the inventive concept can be implemented in various ways. The invention and its embodiments are not limited to the example embodiments described above but may vary within the scope of the claims.

What is claimed is:

1. A measurement system for measuring a moving web of tissue paper, wherein the measurement system comprises
an optical sensor being configured to perform optical transmission measurements of absorption of said moving web within at least two of the following ranges of the measurement system: equal to or shorter than 1.4 μm, 1.6 to 1.9 μm, and equal to or longer than 2.15 μm, which are configured to cover least one absorption characteristic of at least one desired material component of web and at least two references for the at least one absorption characteristic, and
the optical sensor comprises an array of detectors, and the optical sensor is configured to spread different spectral elements of the infrared light to different detectors and is configured to enable detection of at least thirteen mutually independent spectral elements that are distributed over the optical ranges of the measurement system regularly or irregularly in a manner independent of both the at least one absorption characteristic and the at least one reference of said at least one desired material component, the optical sensor being, in that manner, configured to distinguish spectral elements associated with the at least one absorption characteristic of the at least one desired material component and the at least one reference from each other;
the detectors are configured to be activated to detect at least one spectral element associated with at least one absorption characteristic and at least two spectral element associated with the at least two references selected as a function of the wavelengths in said optical range based on the desired material components;
the optical sensor is configured to traverse across the moving web in a transverse direction of the web; and
a data processing unit is configured to process data on at least three of at the at least thirteen independent spectral elements, and form information on at least one of the following based on the data on the at least three independent spectral elements: a basis weight of the web and a weight per unit area of at least one of the at least one desired material component of the web.

2. The system of claim 1, wherein the optical sensor is configured to enable simultaneously the detections of the at least thirteen independent spectral elements that are non-overlapping.

3. The system of claim 1, wherein the optical sensor is configured to measure at least one spectral element associated with the at least one absorption characteristic of the at least one desired material component of the web that comprises one or more solid material components in a fiber form; and
the data processing unit is configured to form basis weight, and a weight per unit area of the at least one solid material component of the web.

4. The system of claim 1, wherein the optical sensor is configured to measure at least one spectral element associated with the at least one absorption characteristic of the desired material components of the web that comprise water, cellulose and one or more ash components; and
the data processing unit is configured to form basis weight, water weight per unit area, cellulose per unit area and at least one weight per unit area of the one or more ash components of the web.

5. The system of claim 3, wherein the optical sensor is configured to measure at least one spectral element associated with the at least one absorption characteristic of the at least one material component that is solid and includes at least one of the following: retention agent material, polymer material, monomer material, filling material, coating material and pigmenting material.

6. The system of claim 4, wherein the optical sensor is configured to measure at least one spectral element associated with the at least one absorption characteristic of the at least one ash component that includes at least one of the following: talcum, kaolin, gypsum, starch, latex, calcium carbonate, titanium oxide.

7. The system of claim 1, wherein the optical sensor is configured to measure at least one spectral element associated with the at least one absorption characteristic of fossil-based polymer fibers, bio-based polymer fibers and/or modified polymer fibers of the web.

8. The system of claim 1, wherein the data processing unit is configured to select detectors of the array in a non-mechanical manner for the detections.

9. The system of claim 8, wherein the data processing unit is configured to activate detectors of the array for the detection and the data processing electrically.

10. The system of claim 8, wherein the data processing unit is configured to process data carried by at least four of at the at least thirteen independent spectral elements.

11. The system of claim 8, wherein the data processing unit comprises one or more processors; and
one or more memories including computer program code;
the one or more memories and the computer program code configured to, with the one or more processors, cause apparatus at least to:
process data on the at least three spectral elements of at the at least thirteen independent spectral elements, and form the information on at least one of the following based on the data on the at least three of the at least thirteen independent spectral elements associated with absorption: the basis weight of the web and the weight per unit area of at least one of the at least one desired material component of the web.

12. A measurement method of a moving web of tissue paper, the method comprising
covering, by an optical sensor comprising an array of detectors, and the optical sensor that spreads different spectral elements of the infrared light to different detectors, at least two of the following optical: ranges of the method: equal to or shorter than 1.4 μm, 1.6 μm to 1.9 μm, and equal to or longer than 2.15 μm, which include at least one absorption characteristic of at least one desired material component of the web and at least two references for the at least one optical absorption characteristic;
enabling detection, by the optical sensor that performs transmission measurements, of at least thirteen independent spectral elements that are distributed over the optical ranges of the method regularly or irregularly in a manner independent of both the at least one absorption characteristic and the at least one reference of said desired material components for enabling the optical sensor to distinguish spectral elements associated with the at least one optical absorption characteristic and the at least two references of the at least one desired material component from each other;
activating the detectors to detect at least one spectral element associated with at least one absorption characteristic and at least two spectral element associated with the at least two references selected as a function of the wavelengths in said optical range based on the desired material components;
performing transversing, the optical sensor, across the moving web in a transverse direction of the web;
processing, by a data processing unit, data on at least three of the thirteen independent spectral elements; and
forming, by the data processing unit, information on at least one of the following based on the data on the at least three independent spectral elements associated with absorption: a basis weight of the web and a weight per unit area of at least one of the desired material components of the web.

13. The method of claim 12, the method further comprising performing a measurement of a multilayer web.

14. The method of claim 12, the method comprising forming baseline curvature of the optical range; and determining the basis weight based on absorptions of the infrared light of the detected elements relating to cellulose and water, and the baseline curvature.
